(19) United States
(12) Patent Application Publication
(10) Pub. No.: US 2011/0071387 A1
(43) Pub. Date: Mar. 24, 2011

(54) MEDICAL IMPLANT DEVICE AND PROBE DEVICE HAVING UP AND/OR DOWN CONVERSION MOLECULES AND METHOD OF MAKING THE SAME

(75) Inventors: Robert J. Petcavich, The Woodlands, TX (US); Murray A. Reicher, Rancho Santa Fe, CA (US)

(73) Assignee: HEALTH BEACONS, INC., Kirkland, WA (US)

(21) Appl. No.: 12/889,619

(22) Filed: Sep. 24, 2010

Related U.S. Application Data
(60) Provisional application No. 61/245,509, filed on Sep. 24, 2009.
(60) Provisional application No. 61/309,582, filed on Mar. 2, 2010.

(51) Int. Cl. A61B 5/05; B05D 3/10; C09K 11/70

(52) U.S. Cl. 600/424; 427/2.3; 264/21

(57) ABSTRACT

A medical device includes a device core and fluorescing molecules attached to the device core. The medical device may be formed in a variety of ways, including providing a medical device and coating at least a portion of the device with a polymer containing the fluorescing molecules, attaching a container containing the fluorescing molecules to a medical device, or molding a medical device from a polymer that includes the fluorescing molecules.

### PRIORITY CLAIM AND CROSS REFERENCE TO RELATED APPLICATIONS

This application claims the benefit of U.S. Provisional Application No. 61/309,582, filed Mar. 2, 2010, and U.S. Provisional Application No. 61/245,509, filed Sep. 24, 2009.

### FIELD

A field of the invention is medical and surgical tools. Example applications of the invention include implantable medical devices and methods of making implantable devices.

### BACKGROUND

Medical care often requires the implantation, passage, or percutaneous insertion of various medical devices such as tubes, catheters, or markers. Identification of such devices requires radiographic evaluation or other forms of medical imaging, such as CT (computed tomography), US (ultrasound), MRI (magnetic resonance imaging), or other methods. Such imaging can be both costly and time consuming. Accordingly, a need exists for non-invasive identification and localization of a broad range of insertable or implantable devices without dependence on pre-existing medical imaging methods. Such insertable or implantable devices may include nasogastric tubes, endotracheal tubes, percutaneously or surgically implanted intravascular catheters, tissue markers, and others, subsequently referred to herein as a “core medical device.”

### SUMMARY OF THE INVENTION

A medical device is provided that includes a device core and fluorescing molecules attached to the device core. The medical device may be formed in a variety of ways.

One embodiment provides a method of constructing a fluorescing medical device including the steps of providing an implantable medical device and coating at least a portion of the implantable medical device with polymer containing fluorescing molecules.

Another embodiment provides a method of constructing a fluorescing medical device including the steps of providing a polymer containing fluorescing molecules and molding the provided polymer to form the medical device.

### BRIEF DESCRIPTION OF THE DRAWINGS

FIG. 1 is a graph showing the absorption coefficient of water, hemoglobin, and oxyhemoglobin for various wavelengths of light;

FIGS. 2(a)-2(c) are diagrams showing the chemical formulae for preferred down-conversion phosphors;

FIG. 2(d) is a graph showing the absorption spectrum of preferred down-conversion phosphors;

FIG. 2(e) is a graph showing the emission spectrum of preferred down-conversion phosphors;

FIG. 3(a) is a perspective view of an example marker of the present invention; and

FIG. 3b is a cross-sectional view of the marker of FIG. 3(a) along the line 3b-3b.

### DETAILED DESCRIPTION OF THE PREFERRED EMBODIMENTS

Fluorescence is a quantum phenomenon in which energy is absorbed by a phosphor as photons of ultraviolet radiation and emitted from the phosphor as photons of visible light. The energy carried by a photon is inversely proportional to the wavelength of the photon. For example, a photon of ultraviolet radiation having a wavelength of 366 nm carries energy of about 3.4 electron volts (eV), while a photon of green light having a wavelength of 520 nm carries energy of about 2.4 eV. Because some energy is lost during fluorescing, it follows then, that in all normal fluorescence, the exciting radiation must have a shorter wavelength than the emitted light. This observation is referred to as Stoke's Law.

Some materials have the ability to absorb multiple photons of long-wavelength infrared light and combine the energies of the absorbed photons to emit a single photon of visible light. These materials are known as up-conversion phosphors, and are referred to as “anti-Stokes” because of their deviation from Stoke's Law. Up-conversion phosphors are a rare class of inorganic crystals. Such up-conversion phosphors have characteristic excitation spectra in the infrared, often coinciding with the wavelengths emitted in infrared light emitting diodes (LEDs) or infrared lasers.

Up-conversion is not as efficient as down-conversion (i.e., normal fluorescence), since multiple photons must be absorbed to create a single up-conversion photon. Accordingly, the source used to excite the up-conversion phosphors must be a relatively high-intensity infrared flashlight or an infrared laser.

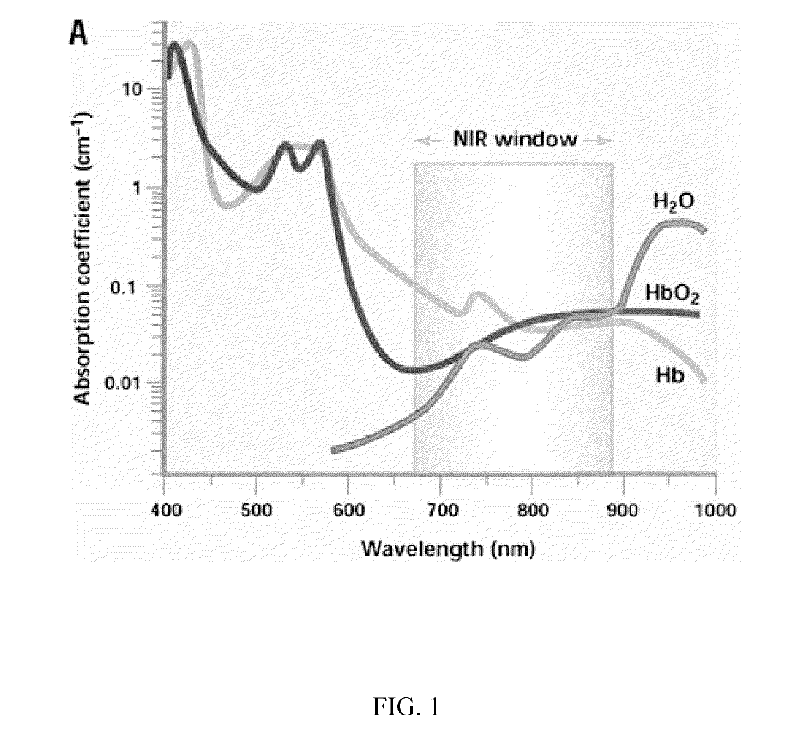

Light in the visible spectrum is partially absorbed by naturally abundant fluorochromes, including hemoglobin. Similarly, infrared light is partially absorbed by water. However, as seen in FIG. 1, the absorption coefficient of water (H₂O), hemoglobin (Hb), and oxyhemoglobin (HbO₂) is relatively low for near-infrared light, when compared to visible light (400-650 nm) or infrared light (>900 nm). Accordingly, near-infrared light penetrates further into the body than visible or infrared light. Specifically, near-infrared photons penetrate approximately 10-15 cm into a body. The phosphors typically fluoresce in a range of about 450 nm to about 850 nm.

Preferred up-conversion phosphors include infrared up-conversion phosphor: green (IRUCG), infrared up-conversion phosphor: blue (IRUCB) and infrared up-conversion phosphor: red (IRUCR) that do not need to be charged with energy prior to up-conversion. Additionally, infrared up-conversion storage phosphor: green (IRSPG) holds energy for a period of time, and releases the energy as photons of green light when excited by a broad range of infrared light. Optical properties of the preferred up-conversion phosphors are shown in Table 1 below.

TABLE 1
Optical Properties of Up-Conversion Phosphors

| | IRUCG | IRUCR | IRUCB | IRSPG |
|---|---|---|---|---|
| Absorption | 948 nm-983 nm | 980 nm | 980 nm | 700 nm-1500 nm |
| Peak | | | | |
| Wavelength | | | | |
| Emission Color | Green | Red | Blue | Green |
| Emission Peak | 552 nm | 620 nm | 445 nm | 490 nm |
| Wavelength | | | | |

The most efficient of the preferred up-conversion phosphors is the IRUCG, followed by IRUCR and IRUCB. Exposing the IRUCG to an infrared flashlight or an infrared laser emitting photons having wavelengths of between about 940 nm and about 980 nm will cause the IRUCG to fluoresce, emitting a small amount of visible light that is difficult to see unless the phosphor is in a very dark room. The IRUCR and IRUCB phosphors will likewise fluoresce under an infrared laser, but detection must be performed by specialized equipment.

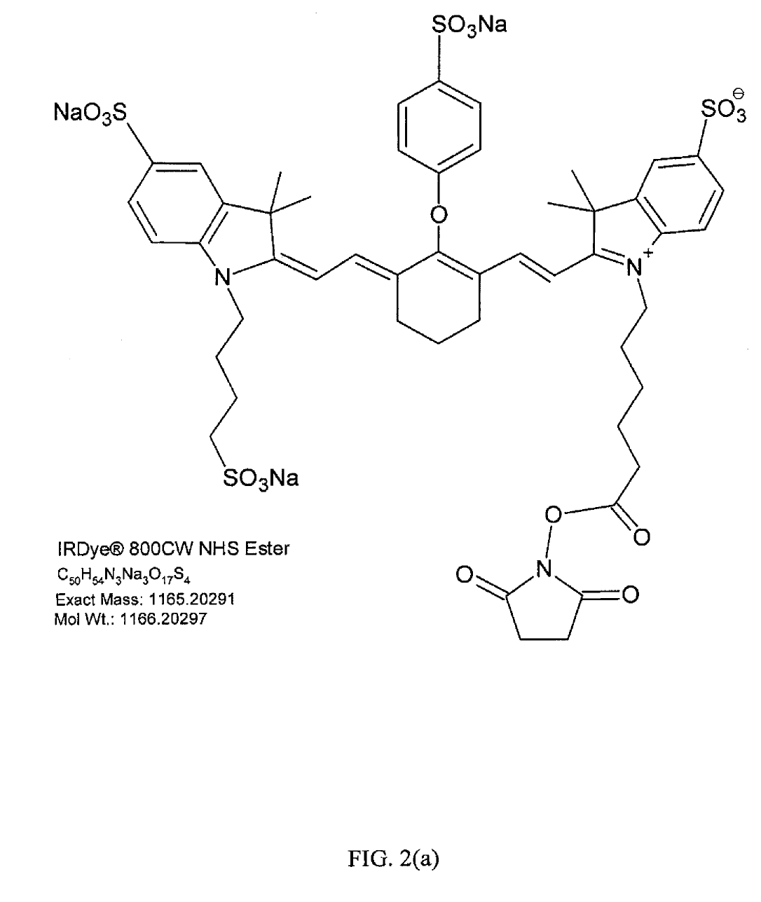

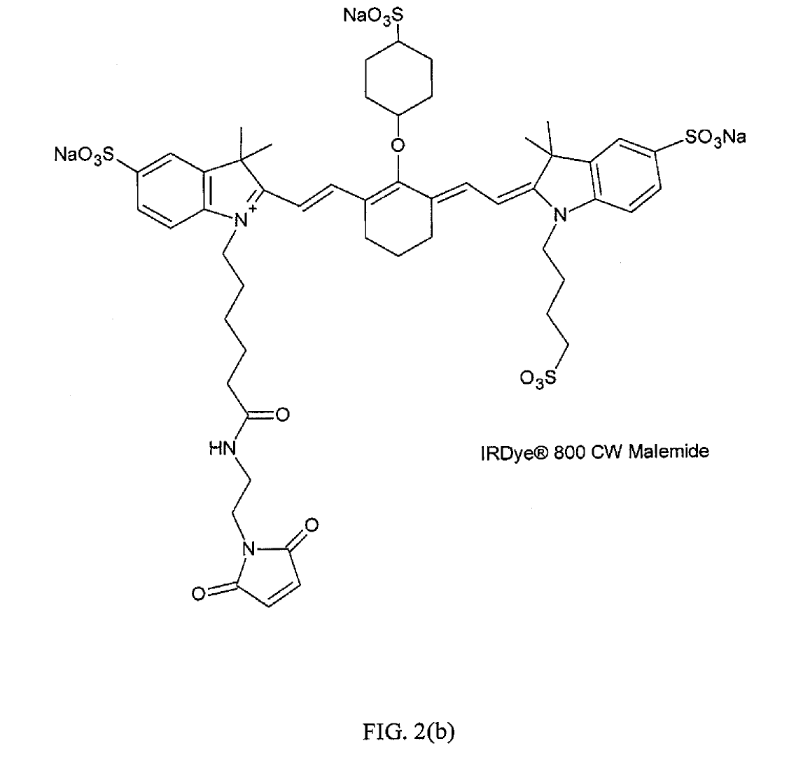

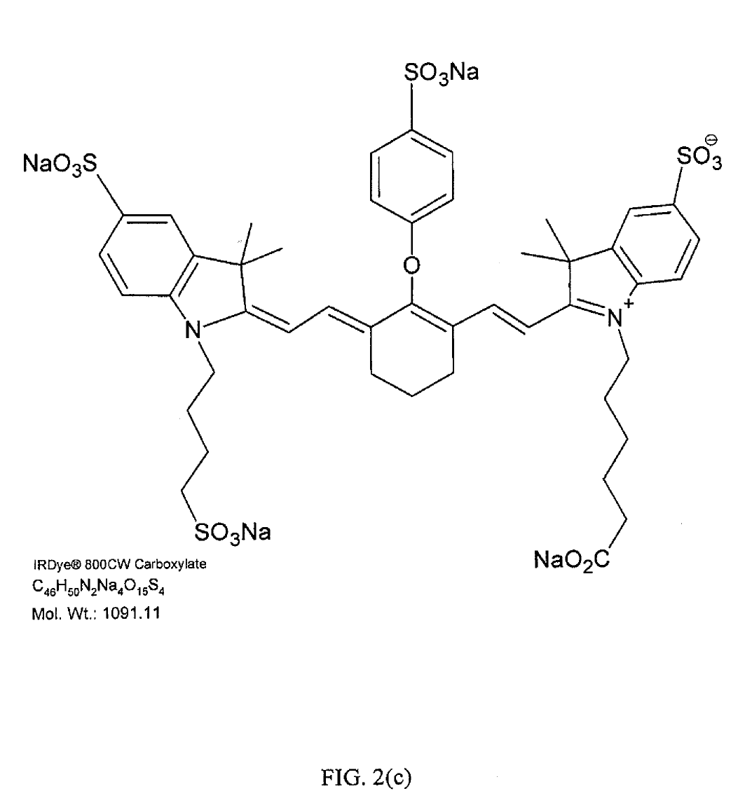

Additionally, down-conversion phosphors (i.e., those that fluoresce normally) can also be used. These down-conversion phosphors behave in a way that is consistent with Stoke's law. Preferred down-conversion phosphors include IRDye® 800CW, which has high solubility in water and a high salt tolerance. The IRDye® can be formed as an NHS ester as shown in FIG. 2(a), a maleimide as shown in FIG. 2(b), and a carboxylate as shown in FIG. 2(c). The IRDye® 800CW most efficiently absorbs light having a wavelength of abut 778 nm, as shown in the absorbance spectrum graph shown in FIG. 2(d). The IRDye® 800CW emits photons having a wavelength of about 790 nm, as shown in the emission spectrum graph in FIG. 2(e).

For purposes of this application, the term “phosphors,” when used on its own, encompasses both up-conversion phosphors and down-conversion phosphors.

The phosphors have a variety of uses, particularly in medical devices such as implantable markers, tubes, catheters, and radio frequency identification tags, as well as medical probes. An implantable device or probe may use phosphors for marking purposes. That is, the phosphors are used to light up the implanted device or probe so that it can be located for resection, maintenance, or the like.

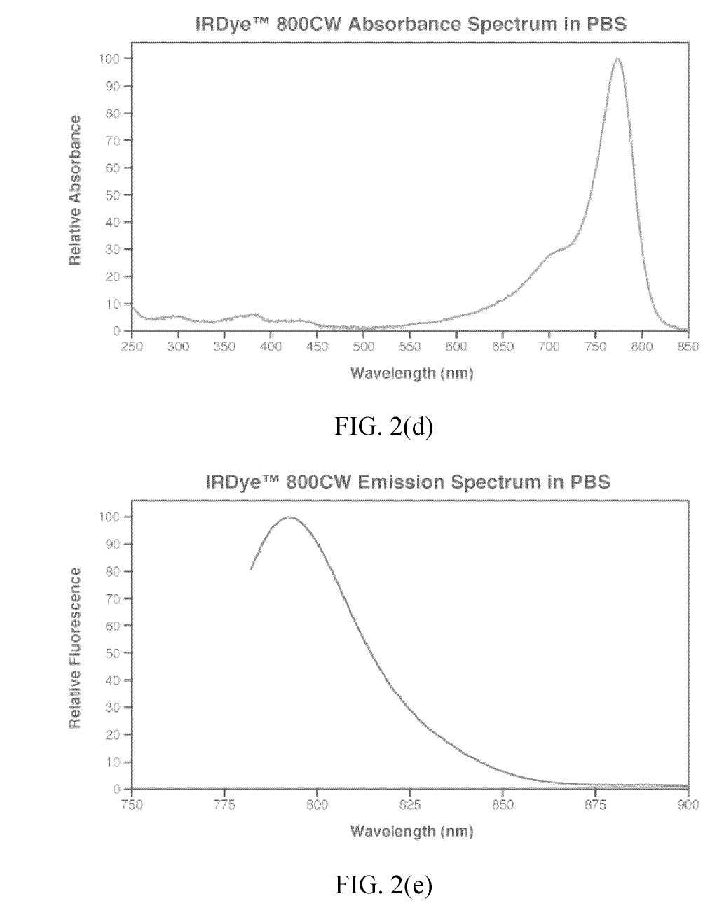

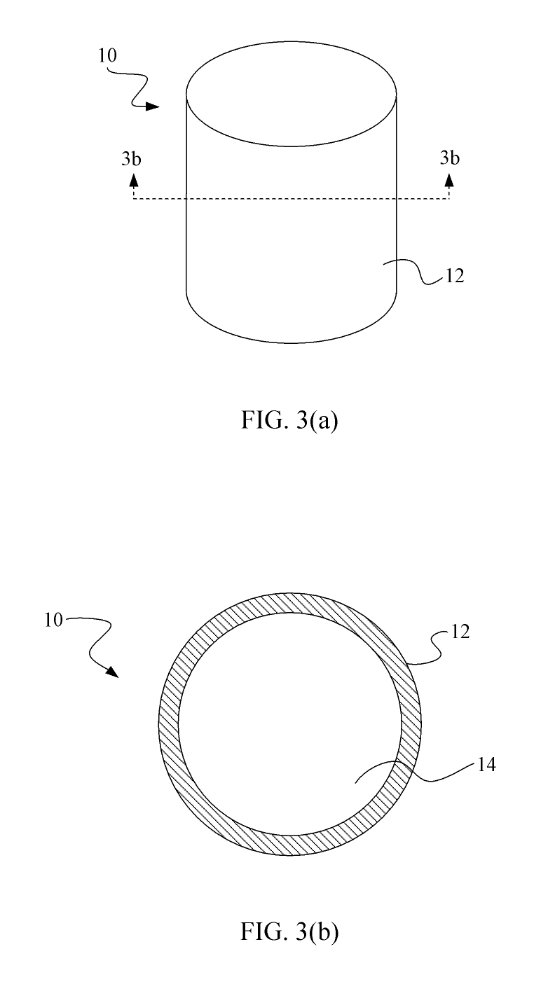

At least a portion of the medical device may be coated with a polymeric material that is approved by the Food and Drug Administration (FDA). Preferred polymers for use in coating portions of the medical devices include polymethylmethacrylate (PMMA), polyethylene (PE), and fluoropolymers such as Teflon, Polysulfone (PS), Polyetherimide (PEI), polylactic acid and its copolymers. However, those of skill in the art will recognize that various other polymers may be used without altering the scope of the invention. As an example, FIG. 3(a) shows a marker 10 that has been entirely coated in polymeric material 12 of the present invention, while FIG. 3(b) shows a cross-sectional view of the marker 10, including a core medical device 14, coated in the polymeric material 12. For simplicity, a marker is shown in FIGS. 3(a) and 3(b). However, those of skill in the art will understand that other medical devices such as a subcutaneous infusion device, an endotracheal or nasogastric tube, or a probe device may be used in place of the marker without departing from the scope of the invention.

The polymeric material 12 is made up of from about 0.1% to about 70% phosphors, with a range of about 3% to about 20% being preferred. The thickness of the polymeric material 12 coated on the medical device 10 is preferably in a range of about 1 μm to about 1000 μm, with a range of about 25 μm to about 199 μm being preferred. The polymeric material 12 is applied to the exterior of the implantable devices using known techniques such as spraying, dipping, or powdercoating. Coating techniques are particularly useful with metal devices, but can also be used plastic devices. If coating a device is inconvenient, a container containing the phosphor may be attached to the implantable device using known attachment means such as a chemical adhesive or the like.

Alternatively, the phosphors may be compounded into FDA approved polymers, and the polymers can be molded into implantable devices using known molding techniques. Preferred polymers for use in molding include polymethylmethacrylate (PMMA), polyethylene (PE), and fluoropolymers such as Teflon, Polysulfone (PS), Polyetherimide (PEI), polylactic acid and its copolymers. Other similar polymers may be used without altering the scope of this invention. When the phosphors are integrated into a polymer compound, the phosphors can make up 90% or less of the polymer compound, by weight, and the phosphors more preferably makes up about 1% to about 20% by weight of the polymer compound.

An exemplary use of the marked implantable devices is a marker used to mark tumor location. Preferably, the marker is, for example, a RFID tag. For example, the markers may be used to mark the location of breast cancer tumors. Specifically, the markers are embedded in target tissue near the tumors to mark the tumor location. The markers are preferably made from titanium, stainless steel, or other inert substances, and are typically sized in a range of about 2 mm to about 4 mm. Intra-operative location of marked tumors often requires pre-operative procedures, such as placement of percutaneous hookwires. Visualization of the marker during the procedure facilitates resection of both the marker and the surrounding target tissue. An illuminating laser emitting light at a wavelength that causes the phosphors used in the marker to fluoresce is integrated into an RFID locator pencil probe. The probe is used to determine a location of the target tissue using a combination of RFID location and fluorescence.

Another example of a use for marked implantable devices is as a marker for subcutaneous infusion catheters. Specifically, use of infusion catheters such as a Port-a-Cath and ventriculo-peritoneal or intra-cranial shunt reservoirs is an increasingly common element of treatment for patients with need of repetitive intravenous infusions. Using phosphors to locate injection sites for the infusion catheters is beneficial for guiding the injection process.

Phosphors are also useful for marking indwelling tubes such as endotracheal and nasogastric tubes. Proper placement of the tubes can be facilitated using localization of a distal tip or another specified segment of the tube. Location of the marked tube segment can be accomplished using percutaneous illumination, particularly in neonatal patients, where distance between the marked tube segment and the outer surface of the skin is relatively small.

Probe devices including needles and surgical probes can also be marked using phosphors. Marked probe devices can be used when locating an implanted marker or other device that is marked with the phosphors.

The phosphors aide surgeons in visualizing a marker or other implanted device within tissue. Further, in instances other than incising tissue to locate a marker or other device, a needle or other probe can be inserted into the tissue to locate the device. In example embodiments, a tip of the needle or probe is marked using the phosphors so the location of the probe tip can be more easily determined when the probe is inserted into the tissue. The needle or probe can be positioned such that the light emitted from the tip of the probe is superimposed with light emitted from a marked target. This method advantageously facilitates non-invasive and precise location of a targeted marked device.

Further, if visual location of the light emitted from the phosphors is impractical, alternative detection means can be used. For example, a photometer configured to measure light intensity at the surface of tissue can be used to locate a targeted marker or other device. A user can scan the photometer over the surface of an area of tissue containing the targeted device to obtain a precise position of the marker based on gathered intensity information.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A method of constructing a fluorescing medical device comprising the steps of:
providing an implantable medical device; and
coating at least a portion of the implantable medical device with polymer containing fluorescing molecules.

2. The method of claim 1, wherein the fluorescing molecules are up-conversion phosphors.

3. The method of claim 1, wherein the polymer contains about 0.1% to about 70% fluorescing molecules.

4. The method of claim 1, wherein the polymer contains about 3% to about 20% fluorescing molecules.

5. The method of claim 1, wherein the coating has a thickness in a range of about 1 μm to about 1000 μm.

6. The method of claim 1, wherein the coating has a thickness in a range of about 25 μm to about 199 μm.

7. The method of claim 1, wherein the core medical device is selected from a group consisting of a marker, a subcutaneous infusion device, an endotracheal or nasogastric tube, a probe device, and a tissue marker.

8. A method of constructing a fluorescing medical device comprising the steps of:
providing a polymer containing fluorescing molecules; and
molding the provided polymer to form the medical device.

9. The method of claim 8, wherein the provided polymer is selected from a group consisting of polymethylmethacrylate, polyethylene, Teflon, polysulfone, polyetherimide, and polylactic acid.

10. The method of claim 8, wherein the fluorescing molecules are up-conversion phosphors.

11. The method of claim 8, wherein the fluorescing molecules account for up to approximately 90% by weight of the provided polymer.

12. The method of claim 8, wherein the fluorescing molecules account for about 1% to about 20% by weight of the provided polymer.

13. The method of claim 8, wherein the medical device is selected from a group consisting of a marker, a subcutaneous infusion device, an endotracheal or nasogastric tube, and a probe device.

14. A medical device comprising:
a device core; and
fluorescing molecules attached to the device core.

15. The medical device of claim 14, wherein the fluorescing molecules are attached to the device core by coating at least a portion of the device core with a polymer containing the fluorescing molecules.

16. The device of claim 14, wherein the fluorescing molecules are contained in a container attached to the device.

17. The device of claim 14, wherein the device core is a medical device selected from a group consisting of a marker, a subcutaneous infusion device, an endotracheal or nasogastric tube, and a probe device.

18. The device of claim 14, wherein the device core and fluorescing molecules are integrally formed from a polymer containing fluorescing molecules.
